(19) United States
(12) Patent Application Publication
(10) Pub. No.: US 2026/0348966 A1
(43) Pub. Date: Nov. 5, 2026

(54) HIGHLY SENSITIVE PLATFORM TO CHARACTERIZE EXTRACELLULAR VESICULAR BIOMARKERS FOR CANCER IMMUNOTHERAPY

(71) Applicants: Ohio State Innovation Foundation, Columbus, OH (US); Spot Biosystems Ltd., Columbus, OH (US)

(72) Inventors: Eduardo Reategui, Columbus, OH (US); Thi Hien Nguyen, Columbus, OH (US); Kwang Joo Kwak, Columbus, OH (US); L. James Lee, Columbus, OH (US)

(21) Appl. No.: 19/740,068

(22) Filed: Jul. 13, 2026

Related U.S. Application Data
(63) Continuation of application No. 18/021,365, filed on Aug. 18, 2021, now Pat. No. 12,724,032.
(60) Provisional application No. 63/067,531, filed on Aug. 19, 2020.

(51) Int. Cl. G01N 33/575; G01N 33/53; G01N 33/543

(52) U.S. Cl. CPC G01N 33/57585; G01N 33/5308; G01N 33/54386; G01N 2333/70596

(57) ABSTRACT

Methods and systems for characterizing extracellular vesicular biomarkers using a biochip with gold nanoparticles. The biochip includes a glass surface, a gold film layer on the glass surface, a plurality of gold nanoparticles coupled to the gold film layer, and a plurality of biotinylated antibodies coupled to the gold nanoparticles. In some implementations, the gold film layer of the biochip is coated with polyethylene glycol (PEG). The biotinylated antibodies are selected to capture specific types of extracellular vesicles. PD-L1/PD-1 proteins and RNAs in extracellular vesicles were characterized for cancer immunotherapy.

### RELATED APPLICATIONS

This application claims the priority benefit of U.S. Provisional Application No. 63/067,531, filed Aug. 19, 2020, entitled “HIGHLY SENSITIVE PLATFORM TO CHARACTERIZE EXTRACELLULAR VESICULAR BIOMARKERS FOR CANCER IMMUNOTHERAPY,” the entire contents of which are incorporated herein by reference.

### BACKGROUND

The present invention relates to systems and methods for characterizing extracellular vesicular biomarkers.

### SUMMARY

In one embodiment, the invention provides a biochip to characterize extracellular vesicular biomarkers for cancer immunotherapy. The biochip includes a glass surface, a gold film layer on the glass surface, a plurality of gold nanoparticles coupled to the gold film layer, and a plurality of biotinylated antibodies coupled to the gold nanoparticles. The biotinylated antibodies are selected to capture specific types of extracellular vesicles. In some implementations, the gold film layer of the biochip is coated with polyethylene glycol (PEG).

Other aspects of the invention will become apparent by consideration of the detailed description and accompanying drawings.

### BRIEF DESCRIPTION OF THE DRAWINGS

FIG. 1 is a flowchart of a method for producing a biochip with gold nanoparticles and for quantifying extracellular vesicular biomarkers using the nanogold biochip.

FIG. 2A is a schematic diagram of a cleaned glass slide used to produce the nanogold biochip of FIG. 1.

FIG. 2B is a schematic diagram of the cleaned glass slide of FIG. 2A after treatment with a binding material that acts as glue between the glass slide and a gold layer.

FIG. 2C is a schematic diagram of the treated glass slide of FIG. 2B after a gold layer is deposited.

FIG. 2D is a schematic diagram of the gold-coated glass slide of FIG. 2C after treatment with a linker solution including biotin-PEG-SH.

FIG. 2E is a schematic diagram of the gold-coated glass slide of FIG. 2D after gold nanoparticles are affixed thereon.

FIG. 2F is a schematic diagram of the gold-coated glass slide of FIG. 2E after incubation with biotinylated antibodies producing a nanogold biochip.

FIG. 3A is a schematic diagram of the nanogold biochip of FIG. 2F after the biotinylated antibodies capture extracellular vesicles.

FIG. 3B is a schematic diagram of the nanogold biochip of FIG. 3A after fluorescent antibodies (or molecular beacons) bind to the captured extracellular vesicles.

FIG. 4A is a series of atomic force microscopic (AFM) images of nanogold biochips with different-sized gold nanoparticles of 5 nm, 30 nm, and 50 nm.

FIG. 4B is a series of total internal reflection fluorescence (TIRF) microscopy images of nanogold biochips with different-sized gold nanoparticles after capturing EVs and the blank nanogold biochips without captured EVs.

FIG. 4C is a series of intensity histograms for each of the TIRF microscopy images of FIG. 4B.

FIG. 4D is a graph of signal-to-noise ratio for different gold nanoparticles sizes based on the TIRF microscopy images of FIG. 4B.

FIG. 5A includes immunofluorescence staining images of EVs produced from H1568 cells with and without interferon-gamma (IFN-γ) stimulation.

FIG. 5B is a graph of the results of an ELISA test for H1568 cells both with and without interferon-gamma (IFN-γ) stimulation.

FIG. 6A is a graph of the size distribution of EVs produced by H1568 cells with interferon-gamma (IFN-γ) stimulation.

FIG. 6B is a graph of the size distribution of EVs produced by H1568 cells without interferon-gamma (IFN-γ) stimulation.

FIG. 7A is a series of TIRF microscopy images of PD-L1 expressions on H1568 EVs with and without interferon-gamma (IFN-γ) stimulation and TIRF microscopy images of the serum alone and a blank control in which PBS is used instead of EVs during the EV capture step.

FIG. 7B is a sequence of intensity histograms for each of the TIRF images of FIG. 7A.

FIG. 7C is a graph of total fluorescence intensity and PD-L1 concentration as a function of EV concentration.

FIG. 7D is a TIRF image of PD-L1⁺ EVs at capturing concentrations of 5×10⁷ EVs/mL.

FIG. 7E is an intensity histogram of the TIRF image of FIG. 7D.

FIG. 8A is a sequence of TIRF images of EVs captured by the nanogold biochip as shown in FIG. 3B for PD-L1 protein and mRNA for cancer patients that respond to immunotherapy, cancer patients that do not respond to immunotherapy, and a healthy donor.

FIG. 8B is a graph of relative fluorescence intensity for the protein captured by the nanogold biochip for non-responders, responders, and healthy donors.

FIG. 8C is a graph of relative fluorescence intensity for the mRNA captured by the nanogold biochip for non-responders, responders, and healthy donors.

FIG. 9 is a graph of relative fluorescence intensity of PD-L1 mRNA captured by the nanogold biochip for a plurality of different patients and healthy donors using different capture antibodies.

FIG. 10 is a sequence of intensity histograms for PD-L1 protein and PD-L1 mRNA captured by the nanogold biochip for healthy donors, non-responders, and responders.

FIG. 11 is a graph of relative fluorescence intensity of PD-L1 protein captured by the nanogold biochip for a plurality of different patients and healthy donors using different capture antibodies.

FIG. 12A is a sequence of TIRF images for PD-L1 protein and PD-L1 mRNA captured by the nanogold biochip using CD63/CD9 as the capture antibodies for healthy donors, non-responders, and responders.

FIG. 12B is a graph of relative fluorescence intensity of PD-L1 protein captured by the nanogold biochip using CD63/CD9 as the capture antibodies for healthy donors, non-responders, and responders.

FIG. 12C is a graph of relative fluorescence intensity of PD-L1 mRNA captured by the nanogold biochip using CD63/CD9 as the capture antibodies for healthy donors, non-responders, and responders.

FIG. 13 is a sequence of intensity histograms for the TIRF images of FIG. 12A.

FIG. 14A is a scatter plot graph of PD-1 mRNA expression level vs. PD-1 m-protein expression level for each plurality of non-responders, responders, and healthy donors.

FIG. 14B is a scatter plot graph of PD-L1 mRNA expression level vs. PD-1 m-protein expression level for each plurality of non-responders, responders, and healthy donors.

### DETAILED DESCRIPTION

Before any embodiments of the invention are explained in detail, it is to be understood that the invention is not limited in its application to the details of construction and the arrangement of components outlined in the following description or illustrated in the following drawings. The invention is capable of other embodiments and of being practiced or of being carried out in various ways.

The immune system responds to cancer via a complex network of cellular interactions in which cytotoxic T-cells, helper T-cells, and natural killer cells are activated and work in concert against tumor cells. However, many metastatic tumors have adopted methods to hijack immune checkpoints to evade immune recognition. One of the recently discovered pathways is the overexpression of programmed cell death ligand 1 (PD-L1) on the surface of tumor cells which binds to programmed cell death protein 1 (PD-1) on T-cells leading to blockade of T-cell activation and protecting tumor cells from T cell-mediated killing. The manipulation of immune checkpoints or pathways by using immune checkpoint inhibitors (ICIs) has emerged as an essential and effective form of immunotherapy and demonstrated successes due to positive and durable clinical trial outcomes. For instance, patients with metastatic melanoma treated with concurrent ipilimumab (anti-cytotoxic T lymphocyte-associated molecule-4 (CTLA-4)) and nivolumab (anti-PD-1) achieved an overall survival rate of 79% at two years. However, the majority of unselected patients do not respond to immunotherapy, for example, the response rate to single-agent PD-1/PD-L1 inhibition in patients with renal cell carcinoma is only 19%. Hence, there is a major challenge and unmet need to determine which individual patients may benefit from PD-1/PD-L1 blockade as well as other immunotherapeutics.

Tumor PD-L1 expression has been approved by FDA as a predictive biomarker for immunotherapy and detected using immunohistochemistry (IHC). Four PD-L1 IHC assays using four different PD-L1 antibodies (22C3, 28-8, SP263, SP142) on two different automated staining platforms (Dako and Ventana) have been registered with FDA. Patients with higher expression of PD-L1 on their biopsies are associated with improved response rates to PD-1/PD-L1 blockade. Following initiation of checkpoint inhibitor therapy, repeating sampling of tumor biopsies is necessary to monitor the response to immunotherapy. Serial tumor biopsies are invasive with potentially serious complications, and the sampling at a single metastatic site may not represent the entire tumor burden in a highly heterogeneous cancer. Hence, there is an unmet need to detect those predictive biomarkers in a non-invasive manner via liquid biopsies such as blood and urine. This approach will help to integrate signals from all metastatic foci and can be repeated serially throughout immunotherapy.

Extracellular vesicles (EVs) are lipid particles released from cells, varying from 50 nm to a few microns and including a) exosomes generated inside multivesicular endosomes, b) microvesicles shed from the plasma membrane, and c) oncosomes differentiated from apoptotic cellular bodies. They contain different cargos, including proteins, RNA, DNA and lipids, which can be trafficked between cells and serve as mediators of intercellular communication. The most common techniques to characterize RNAs and proteins in EVs are polymerase chain reaction (PCR), next-generation sequencing (NGS), mass spectroscopy (MS), western blot, enzyme-linked immunosorbent assay (ELISA), and flow cytometry. However, these existing methods primarily focus on bulk analysis, which requires many EVs with limited resolution and sensitivity. Compared to abundant biomarkers in EVs (like tetraspanins CD9, CD63, CD81, etc.), quantification of low-abundant biomarkers is challenging. There have been some efforts to characterize PD-1/PD-L1 proteins and PD-L1 RNA from EVs using western blot, flow cytometry and PCR; however, to the best of our knowledge, there have been no studies co-quantify these molecular contents (proteins and RNAs) from EVs up to now. The combination of these molecular contents would provide a more comprehensive profiling of EVs from cancer patients.

We have recently developed biochip platforms to isolate tumor EVs specifically and sensitively detect EV-encapsulated target RNAs using cationic lipoplex nanoparticles (CLNs) containing target-specific molecular beacons (MBs). These technologies have been shown to successfully capture and characterize bulk EVs from different cancers and non-cancerous conditions. However, to quantify low-abundant biomarkers for immunotherapy, a more sensitive platform that enables on-chip single EV characterization is needed. In this work, we aim to develop a highly sensitive immunoaffinity-based platform for co-quantification of PD-1/PD-L1 proteins and mRNAs in EVs derived from non-small cell lung cancer (NSCLC) patient blood. The high sensitivity is achieved by characterization at the single EV level due to a combination of a biochip which maximizes signal-to-noise ratio (SNR), and a high-resolution total internal reflection fluorescence (TIRF) microscopy. The biochip was made of a glass coverslip coated with polyethylene glycol (PEG) to prevent non-specific bindings and gold spherical nanoparticles (NPs) to amplify signals and improve sensitivity. Nanoparticles (e.g., cationic lipoplex nanoparticles and gold nanoparticles) are nanoscale particles of matter between 1 and 100 nanometers in diameter. Different antibodies were tethered on the chip surface to capture and sort EVs into subpopulations based on their membrane protein compositions. PD-1/PD-L1 antibodies with a tyramide signal amplification (TSA) technology were then used to quantify the corresponding membrane protein contents on the captured single EVs. CLNs with transcript-specific probes tagged with MBs were also fused with the captured individual EVs to identify and quantify PD-1/PD-L1 mRNA in the EVs. A sensitive automated TIRF microscope was used to detect and quantify specific biomolecules based on fluorescence intensity.

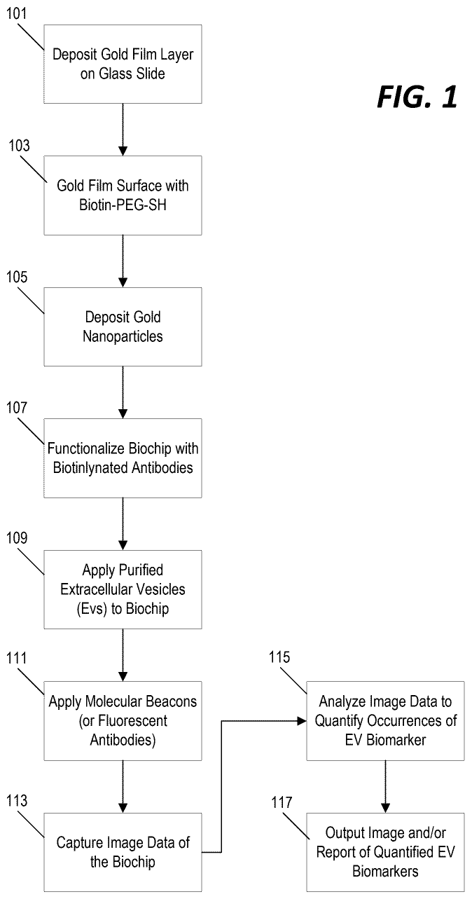

FIG. 1 illustrates a method of producing a biochip and using that biochip for total internal reflection fluorescence (TIRF) microscopy. As shown in various examples described herein, high-resolution TIRF microscopy can be used in cell biology for the visualization of complex processes or the composition of single objects due to its high signal-to-noise ratio (SNR) and the capability to detect single molecules. This imaging technique restricts excitation to a precise focal plane near the coverslip and eliminates out-of-focus fluorescence, thereby allowing single-molecule detection. As also demonstrated by these examples, high-resolution TIRF microscopy can be applied to visualize extracellular vesicles (EVs) pre-stained with a fluorescent membrane dye on a glass slide. However, for on-chip EV labeling, non-specific binding of labeling antibodies to the glass coverslip can make it difficult to differentiate true binding events from the background noise. To prevent non-specific binding of biomolecules to the glass surface, the example of FIG. 1 produces and utilizes a biochip coated with polyethylene glycol (PEG) and gold nanoparticles.

In the method of FIG. 1, a gold film layer is deposited on a glass slide (step 101). In some implementations, for example, the gold film layer is coupled to the glass slide by activating the cleaned cover glass using a UV-ozone cleaner (such as, for example, UVO Cleaner Model 42, Jelight, Irvine, CA) and then modifying with the vapor of (3-mercaptopropyl) trimethoxysilane (MPTMS, Sigma-Aldrich, St. Louis, MO) for 10 minutes in a low-pressure (e.g., ~0.1 Torr) vacuum chamber. The MPTMS layer then serves as a glue layer for the deposition of a 12-nm-thick gold (Au) layer using a Denton e-beam evaporator (DV-502A, Moorsetown, NJ). In another example, the thin gold layer is deposited via titanium (thickness ~2 nm), which serves as a “metal glue” on the glass coverslip.

The gold film surface is then coated with PEG (step 103) and streptavidin-conjugated gold nanoparticles (step 105). In one specific example, the freshly prepared Au-coated glass may be transferred to a linker solution containing 1-thiahexa(ethyleneoxide) lipidic anchor molecule WC14 [20-tetradecyloxy-3,6,7,12,15,18, 22-heptaoxahexa-tricontane-1-thioil], a lateral spacer β-mercaptoethanol (βME, Sigma-Aldrich), and biotin-PEG-SH (Nanocs, New York, NY) (molar ratio=30:69:1) in 200 proof ethanol (Fisher Scientific) for 16 hours at room temperature in the dark. In some implementations, the glass coverslip is then rinsed with ethanol to remove excess mixture physically adsorbed on the surface and air-dried. The treated glass is then attached to a 64-well tray (Grace Bio-Labs ProPlate tray set, Sigma-Aldrich) and washed thoroughly with DI water. Next, 0.01% (w/v) streptavidin-conjugated gold nanoparticles (NPs, Nanocs Inc.) in PBS are applied into the wells for 2 hours at room temperature on a rocker at 24 RPM. As described in further detail below, different sizes of gold nanoparticles may be used, and, in some implementations, the size of the gold nanoparticles may be selected to achieve a target EV capture efficacy and/or non-specific binding of antibodies.

After the gold nanoparticles are deposited, the biochip is functionalized with biotinylated antibodies (i.e., antibodies that have undergone a process of biotinylation in which biotin is attached to the antibodies) to capture a specific EV subpopulation (step 107). In some implementations, the biochip is first rinsed three times with PBS before the surface is incubated with capture antibody cocktails overnight at 4 °C on the rocker. In some implementations, for PD-L1 protein detection, a cocktail with 20 μg/mL each of recombinant chimeric EGFR monoclonal antibody (Cetuximab, Erbitux, ImClone LLC, Branchburg, NJ), a goat EpCAM/TROP-1 polyclonal antibody (#AF960, R&D Systems, Minneapolis, MN), and a goat ErbB2/Her2 polyclonal antibody (#AF1129, R&D Systems) is used. In some implementations, for PD-1 protein and PD-1/PD-L1 mRNA detection, 20 μg/mL each of a mouse CD63 monoclonal antibody (#sc-5275, Santa Cruz Biotechnology, Dallas, TX) and a mouse CD9 monoclonal antibody (#MAB1880, R&D Systems) was chosen as a capture antibody cocktail. In some implementations, these antibodies are biotinylated using an EZ-Link micro Sulfo-NHS-biotinylation kit (ThermoFischer Scientific, Waltham, MA) before the incubation. In some implementations, the device is washed three times on the next day with PBS and then blocked 3% (w/v) BSA (Sigma-Aldrich) and 0.05% (v/v) Tween-20 (Sigma-Aldrich) in PBS for 1 hour at room temperature before EV capture. In other implementations, this blocking is performed before and/or after EV capture to minimize non-specific binding further.

After the biochip is produced and functionalized with biotinylated antibodies, purified extracellular vesicles (EVs) are applied to the biochip (step 109). EVs selectively bind to the antibodies at the gold nanoparticles and molecular beacons (or other fluorescent antibodies) are then applied to bind with the captured EVs (step 111). Image data of the biochip is then captured, for example, using TIRF microscopy (step 113) and the captured image data is processed/analyzed to quantify occurrences of the EV biomarker (step 115). The TIRF image and/or report data of the quantified EV biomarkers is then output on a system display and/or stored in memory for later use (step 117).

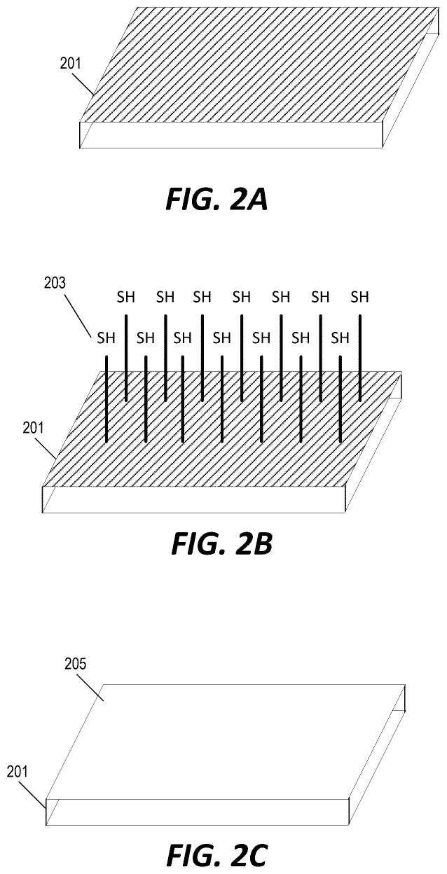

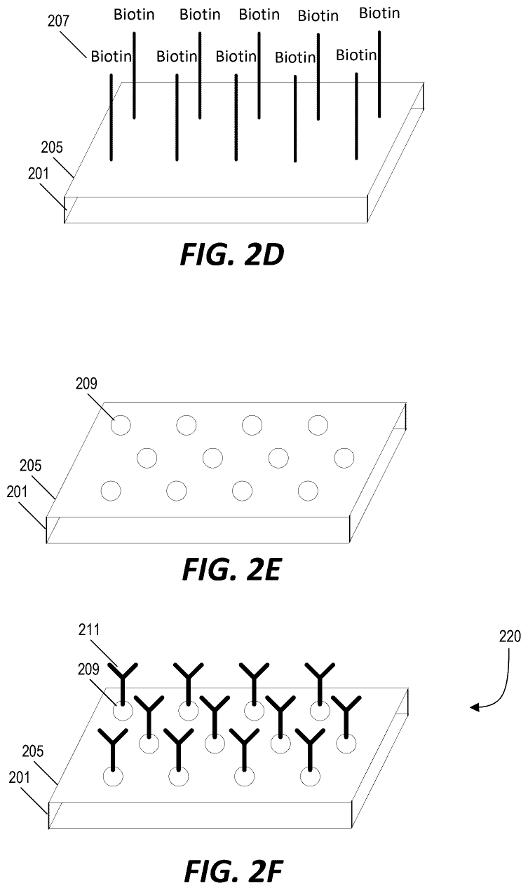

FIGS. 2A through 2F provide schematic illustrations of the biochip at various stages of the fabrication process (e.g., step 101 through step 107 in the method of FIG. 1). FIG. 2A shows the cleaned glass slide 201. After treating the slide 201 with the MPTMS, -SH mercapto groups 203 are bound to the surface of the glass slide 201 as shown in FIG. 2B, which, in turn, serves as the “glue” for coupling the thin gold layer 205 to the glass slide 201 as shown in FIG. 2C. Although MPTMS is shown in the example of FIG. 2B, in other implementations, different binding mechanisms may be used for coupling the gold layer to the glass slide. For example, as discussed above, titanium may be used instead as a “metal glue” for attaching the gold layer 205 to the glass slide 201. FIG. 2D shows the biotin layer 207 applied to the surface of the gold layer 205 on the glass slide 201 and FIG. 2E shows the gold nanoparticles 209 deposited on the surface of the gold layer 205. Finally, FIG. 2F shows the biochip 220 functionalized with the biotinylated antibodies 211 coupled to the gold nanoparticles 209. Although not illustrated in the example of FIGS. 2A through 2F, in some implementations, the functionalized biochip is attached to a structure configured to divide the surface area of the biochip into different chambers. For example, in some implementations, the biochip is attached to a silicone gasket with 64 chambers (Grace Bio-Labs, ProPlate tray set, Sigma-Aldrich).

The thin gold coating improves the SNR of TIRF microscopy through the surface plasmon resonance (SPR) effect, which takes place when total internal reflection occurs at a metal film-liquid interface. A biochip coated with a thin gold film and PEG is able to sensitively quantify target RNAs within EVs in bulk for non-invasive early cancer diagnosis. As discussed in further detail in the examples below, the inclusion of gold nanoparticles on top of the thin gold coating layer further affects the performance of the biochip in capturing EVs.

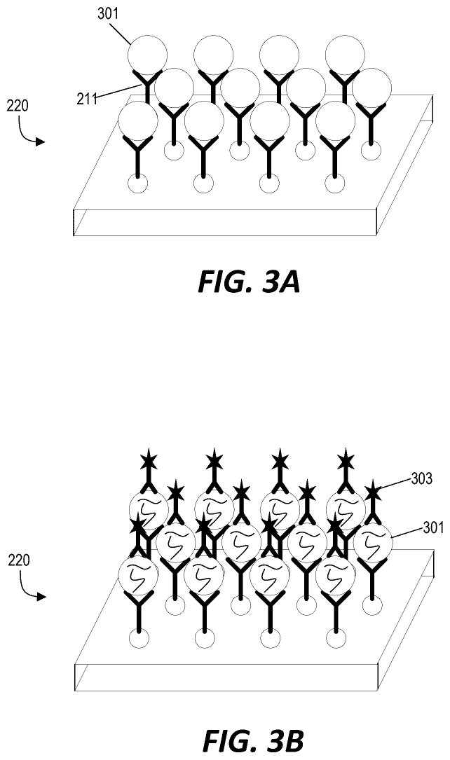

FIGS. 3A and 3B schematically illustrate an example of using the biochip 220 for capture and visualization of target EVs. The functionalized biochip 220 is exposed to a sample (e.g., a biofluid) and, as shown in FIG. 3A, target EVs 301 from the sample bind to the antibodies 211 on the biochip 220. After the EVs 301 are captured by the biochip 220, fluorescent antibodies or molecular beacons 303 bind to the captured EVs 301. When TIRF images of the biochip 220 are captured, the molecular beacons 303 are visible in the captured image data. The number and/or intensity of the molecular beacons 303 visible in the captured TIRF images can then be used to characterize and quantify various aspects of the sample related to presence and/or concentrations of particular EVs.

In some of the examples described herein, the surface topography of devices coated with different sizes of streptavidin-conjugated gold NPs may be characterized using an atomic force microscope (Asylum Research MFP-3D-BIO AFM, Oxford Instruments, Abingdon, United Kingdom). Before imaging, the devices were rinsed thoroughly with deionized (DI) water to avoid salt crystals and then air-dried.

H1568 cells (NCI-H1568, ATCC® CRL-5876™, Manassas, VA) were cultured in a growth medium containing RPMI 1640 (ThermoFisher Scientific), 10% (v/v) fetal bovine serum (FBS, Sigma-Aldrich) and 1% (v/v) penicillin-streptomycin (PS, ThermoFisher Scientific). The medium was replaced every 2 to 3 days, and cultures were maintained in a humidified incubator at 37 °C with 5% CO2. When the cells reached 80%-90% confluence, they were detached using TrypLE™ express enzyme (ThermoFisher Scientific) and passaged at 1:3-1:6 ratios. H1568 cells at passages 6-10 were used in this study.

For PD-L1 staining of cells, H1568 cells were seeded at a density of 105 cells/mL in 16-well chambers (Grace Bio-Labs ProPlate® tray set) attached to a glass slide (Fisher Scientific). To stimulate PD-L1 expression, the cells were incubated with 100 ng/ml recombinant human IFN-γ (Peprotech, Rocky Hill, NJ) in the growth medium for 48 hr. The cells without IFN-γ stimulation were employed as a control. After that, they were fixed in 10% (v/v) formaldehyde (Fisher Scientific) for 15 min at RT and then permeabilized with ice-cold 100% methanol for 10 min at −20 °C. A blocking buffer containing 5% (v/v) normal goat serum (ThermoFisher Scientific) and 0.3% (v/v) Triton X-100 (Sigma-Aldrich) in phosphate-buffered saline (PBS) was subsequently applied to the samples for 1 hr at RT. Rabbit PD-L1 monoclonal antibody (#86744S, Cell Signaling Technology, Danvers, MA) was diluted 200-fold in an antibody dilution buffer (1% (w/v) BSA and 0.3% (v/v) Triton X-100 in PBS) and then incubated with the cells overnight at 4 °C. The next day, the cells were rinsed three times in PBS for 5 min each and subsequently incubated with a goat anti-rabbit IgG (H+L) secondary antibody—Alexa Fluor®647 conjugate (Cell Signaling Technology) at a dilution of 1:500 in the antibody dilution buffer, for 1 hr in the dark at RT. After washing three times with PBS for 5 min each, the glass slide was detached and mounted onto a cover glass (Fisher Scientific) using ProLong™ Gold Antifade Mountant with DAPI (ThermoFisher Scientific). The images were taken using a fluorescence microscope (Nikon Eclipse Ti2, Nikon, Melville, NY).

For EVs purification from cell culture supernatants, H1568 cells were first grown to 80% confluency in the growth medium, then washed with PBS and changed to an RPMI medium supplemented with 10% (v/v) EVs-depleted FBS, 1% (v/v) PS and 100 ng/mL IFN-γ for 48 hr. EVs isolated from the medium without IFN-γ supplement were used as controls. EVs-depleted FBS was the filtrate produced from FBS through tangential flow filtration (TFF) with a 500 kDa molecular weight cut-off (MWCO) hollow fiber filter (polysulfone, Repligen, Waltham, MA). After collection, the culture supernatants were centrifuged at 1000 rpm for 2 min (Centrifuge 5810R, Eppendorf, Hauppauge, NY) to remove cell debris.

Blood samples were obtained with informed consent from healthy donors and cancer patients using an approved Institutional Review Board protocol at The Ohio State University. Blood samples from stage IV NSCLC patients were collected before they underwent immunotherapy. Serum was separated from blood using a BD Vacutainer™ SST™ Serum Separation Tube (#367985, Fisher Scientific) according to the manufacturer's protocol. 150 μL serum was diluted to 50 mL with PBS before purification.

The prepared cell supernatants and sera were firstly filtered through 1 μm filters (GE Healthcare Whatman™ Puradisc GMF, Fisher Scientific). They were subsequently concentrated and diafiltrated using TFF with the 500 kDa filter for purification. After TFF, the retentates were concentrated to 1 mL using centrifugal units (10 kDa MWCO, MilliporeSigma™ Amicon™ Ultra Centrifugal Filter Unit, Fisher Scientific) at 3000×g for 20 min. The concentration of EVs were quantified using a tunable resistive pulse sensing (TRPS) technology (qNano Gold instrument, Izon Science, Medford, MA) with NP150 (target size range 70-420 nm) and NP600 (target size range 275-1570 nm) nanopore membranes.

For CD63 detection of EVs, EVs produced from H1568 cells without IFN-γ stimulation were adjusted to a concentration of 1010 particles/mL. Thereafter, 20 μL purified EVs were applied onto devices coated with different NP sizes. PBS was used as a blank control. The following incubation and washing steps were performed at RT on the rocker. EVs were captured for 2 hr, washed three times with PBS, and then blocked with 3% (w/v) BSA and 0.05% (v/v) Tween® 20 in PBS for 1 hr. The samples were subsequently incubated with a mouse CD63 monoclonal antibody (MX-49.129.5)-Alexa Fluor® 488 conjugate (#sc-5275 AF488, Santa Cruz Biotechnology) at a dilution of 1:200 in 1% (w/v) BSA in PBS for 1 hr. Next, the devices were rinsed three times with 0.05% (v/v) Tween® 20 in PBS, and their images were taken using a TIRF microscope (Nikon Eclipse Ti Inverted Microscope System). The images were recorded by an Andor iXon EMCCD camera with a 100x oil lens at the same laser power and exposure time. For each sample, 100 (10×10 arrays) images were collected.

For PD-1/PD-L1 protein detection of EVs, 20 μL purified EVs from H1568 cells (with and without IFN-γ stimulation) and blood samples (healthy donors and cancer patients) were captured onto devices coated with 30 nm streptavidin-conjugated gold NPs for 2 hr. PBS was used as a blank control, and also a washing buffer. All incubation and washing steps were conducted at RT on the rocker. After capture, the samples were rinsed three times and stained for PD-1/PD-L1 proteins using an Alexa Fluor™ 647 Tyramide SuperBoost™ kit (#B40926, ThermoFisher Scientific). Firstly, the EVs were fixed with 10% (v/v) formaldehyde for 10 min. After washing, 3% Hydrogen Peroxide Solution was added to quench the endogenous peroxidase activity of the samples for 15 min, followed by incubation with 3% (w/v) BSA and 0.05% (v/v) Tween® 20 in PBS for 1 hr. The PD-L1 antibody (diluted 500-fold in Blocking Buffer) or rabbit PD-1 monoclonal antibody (#86163S, Cell Signaling Technology, diluted 1000-fold in Blocking Buffer) was then diluted incubated for 1 hr. Next, the samples were washed three times for 10 min each before incubation with a poly-HRP-conjugated secondary antibody for 1 hr. After washing three times for 10 min each, a Tyramide Working Solution was applied for 10 min. The reaction was stopped using a Reaction Stop Reagent. Thereafter, the samples were rinsed three times and imaged using the TIRF microscope as mentioned above.

Molecular beacons (MBs) (listed 5′-3′) targeting PD-1 and PD-L1 mRNAs used in this study were +GGT +CCT /iCy3/ +CCT +TCA +GGG GCT GGC GCC CCT GAA GG /BHQ_2/ and +GGT +AGC /iCy3/ +CCT +CAG +CCT GAC ATG AGG CTG AGG /BHQ_2/, respectively. They were designed based on NCBI reference sequence of PD-1 (NM_005018.3) and PD-L1 (NM_014143.4) using Primer3 and BLAST (Primer-BLAST) provided by NCBI-NIH. Locked nucleic acid (LNA) nucleotides (positive sign (+) bases) were incorporated into oligonucleotide strands to improve the thermal stability and nuclease resistance of MBs for incubation at 37 °C. The designed MBs were custom synthesized and purified by Sigma-Aldrich. An aqueous solution of MBs in PBS was vigorously mixed with a lipid formulation of DOTAP, Cholesterol, POPC and PEG-DSPE in 200 proof ethanol, and then sonicated for 5 min using an ultrasonic bath. The MB/lipid mixture was subsequently injected into PBS, vortexed and sonicated for 5 min. Finally, it was dialyzed with 20 kDa MWCO to remove free MBs.

For PD-1/PD-L1 mRNA detection of EVs, 20 μL purified EVs from serum samples of healthy donors and cancer patients were captured onto the 30 nm-nanogold chips for 2 hr at RT. After washing with PBS, PD-1/PD-L1 CLN MBs were applied and incubated for 2 hr at 37 oC. The samples were finally rinsed with PBS and imaged using the TIRF microscope.

For analysis of the captured TIRF images, all the spots in the TIRF image were firstly located with distinguished edges, and background noise was removed by Wavelet denoising method using Matlab. Net fluorescence intensity of the spot was then calculated by subtracting the mean intensity of pixels in the spot to the mean intensity of pixels surrounding the spot. Subsequently, histograms of net fluorescence intensities of all the spots were obtained and their total fluorescence intensity was also calculated.

For ELISA testing described in the examples below, PD-L1 expression levels in H1568 cells (with and without IFN-γ stimulation) and on the surface of H1568 EVs (with IFN-γ stimulation) were quantified using a PD-L1 Human ELISA kit (#BMS2212, ThermoFisher Scientific). For the cells, they were lysed in RIPA buffer (ThermoFisher Scientific) with the addition of Thermo Scientific™ Halt™ Protease and Phosphatase Inhibitor Cocktails on ice for 5min, and then centrifuged at 14,000×g for 15 min to remove cell debris. For the EVs, they were spiked in healthy donor serum at different concentrations ranging from 0 to 1011 particles/mL. All the samples were subsequently incubated in the ELISA plate and their PD-L1 expressions were quantitatively detected according to the manufacturer's instructions. The PD-L1 concentration in the cell lysis was normalized to its total protein concentration, which was measured using a Pierce™ Rapid Gold BCA Protein Assay kit (ThermoFisher Scientific).

All in vitro experiments and assays were repeated at least three times. All clinical samples were repeated two times. Sigma Plot 14, JMP Pro 14 and Matlab R2019a were used for data analysis. The data were expressed as mean ±SD and compared by Student's t-test. A p-value below 0.05 was considered statistically significant.

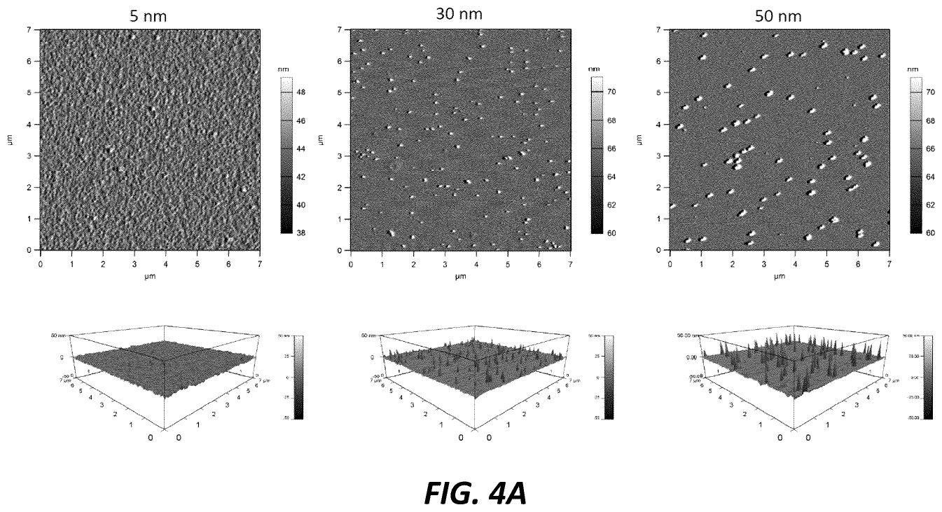

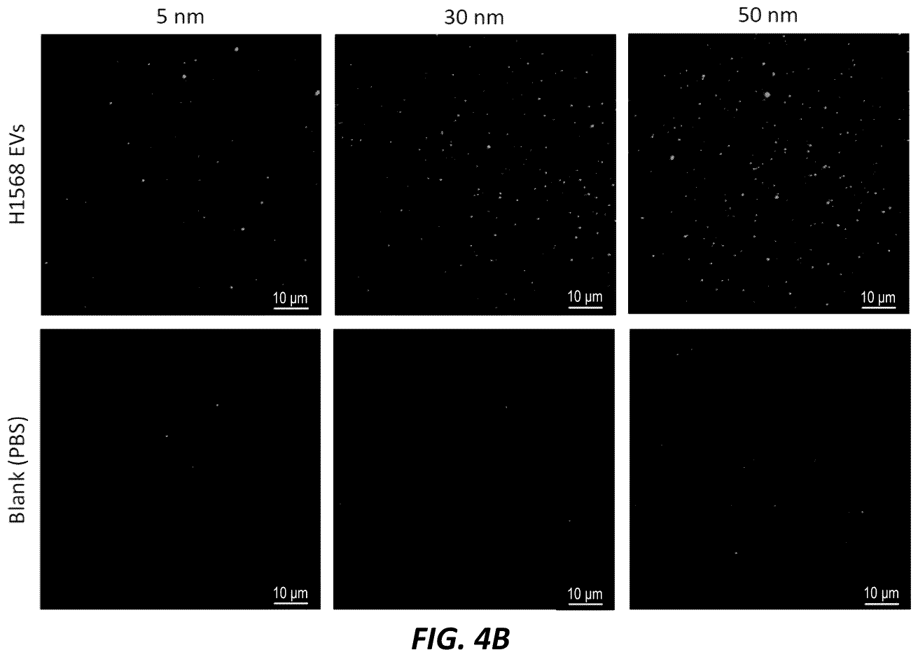

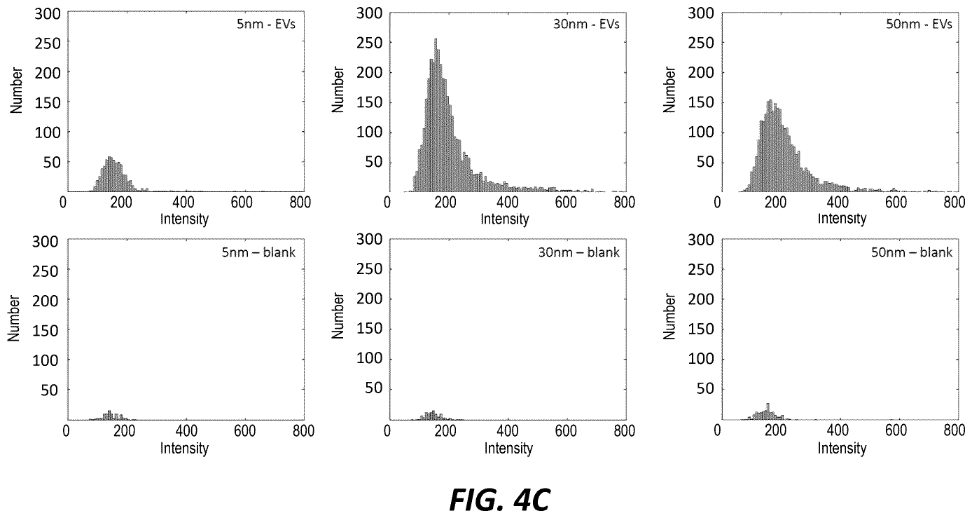

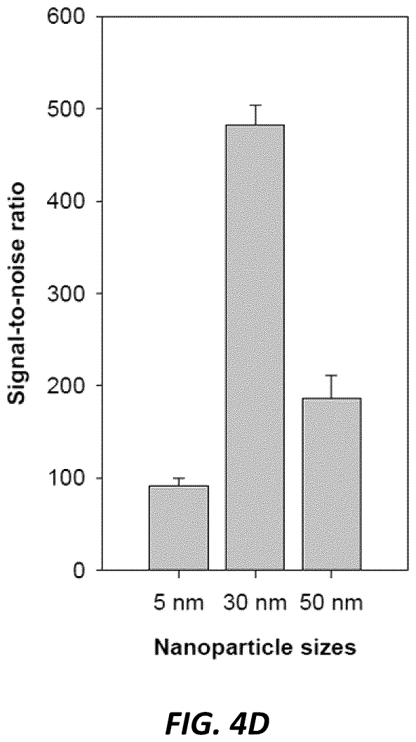

Using the systems and methods described above, the size effect of streptavidin-conjugated gold NPs (5; 30 and 50 nm) on SNR of our platform was investigated. Atomic force microscopic (AFM) images showed that the NPs of all sizes were uniformly dispersed on the devices (FIG. 4A). With the same concentration loaded to each well (0.01% (w/v) based on gold), it is logical that the number of NPs coated on the devices decreased with the particle size. H1568, an NSCLC cell line, was used to evaluate our nanogold chip. Their purified EVs were captured on the chip using a cocktail of epidermal growth factor receptor (EGFR), epithelial cell adhesion and activating molecule (EpCAM) and human epidermal growth factor receptor 2 (Her2) biotinylated antibodies. We previously demonstrated that this cocktail facilitated the capture of a heterogeneous population of CTCs compared to single antibodies. Cluster of differentiation 63 (CD63) staining of the captured EVs were then used to evaluate the effect of NP sizes on fluorescence signal intensity and background noise. TIRF images and their histograms revealed that devices coated with 30-nm gold NPs had higher signal and lower background, while both signal and background were lower for those coated with 5-nm NPs and higher for those coated with 50-nm NPs (FIGS. 4B and 4C). Among these sizes, 30 nm was shown to produce the highest SNR (p<0.001, FIG. 4D). Therefore, 30-nm gold NPs were chosen for our subsequent experiments. Metal nanoparticles exhibit localized SPR, which can be seen by a strong UV-Vis absorption band that is absent from bulk metal. Gold NPs are considered to be better than other NPs for signal enhancement because they can cause higher refractive index (RI) changes and resonance angle shift. The size of gold NPs have been known to significantly affect the level of signal enhancement due to changes in surface coverage and RI. Larger NPs resulted in greater RI shift. In cancer biomarker detection using SPR biosensor, 20-nm gold NPs reduced the LOD 8 times, while 40-nm gold NPs lowered the LOD 65 times with respect to the sandwich assay. By measuring the SPR angle shift in the presence of aqueous methanol, 30 nm was found to be the optimal particle size compared to 10 and 60 nm. In another study to develop a highly sensitive nuclease-linked fluorescence oligonucleotide assay, among four different sizes tested (13; 30; 40 and 50 nm), the 40-nm gold NPs yielded the strongest signal. As such, the optimal particle size varies with devices, and in our case, it was 30 nm.

### In Vitro PD-L1 Characterization

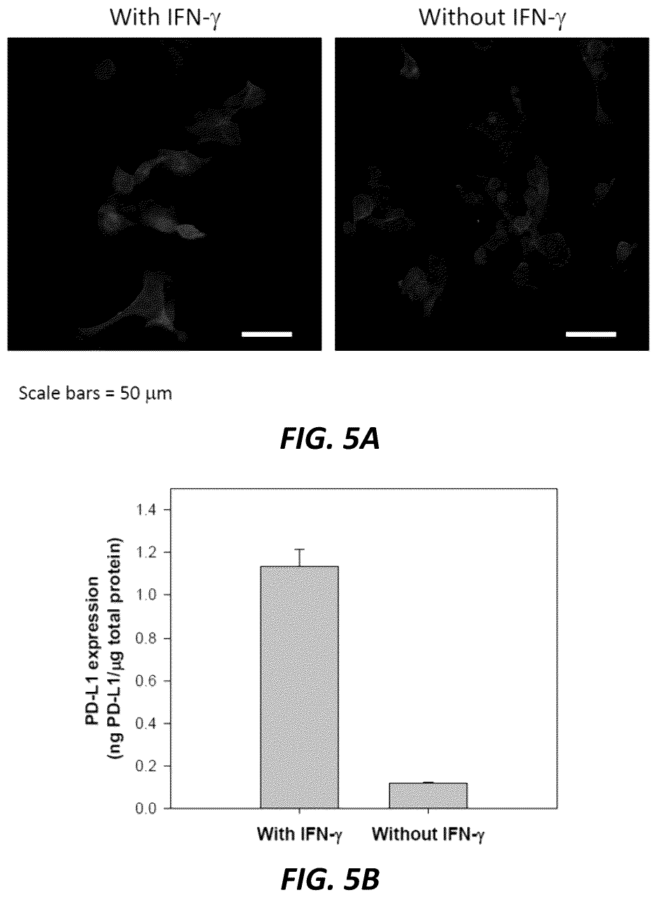

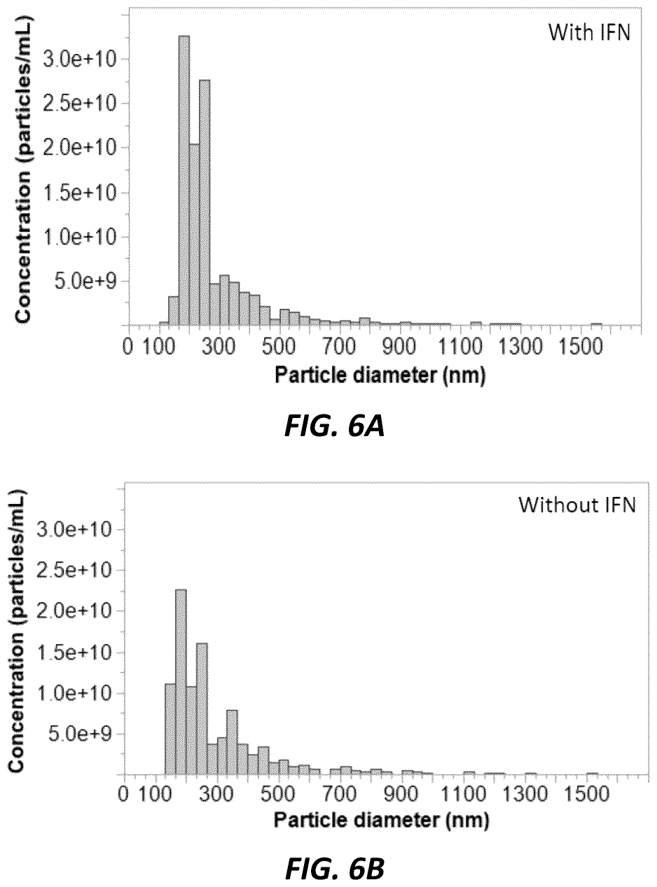

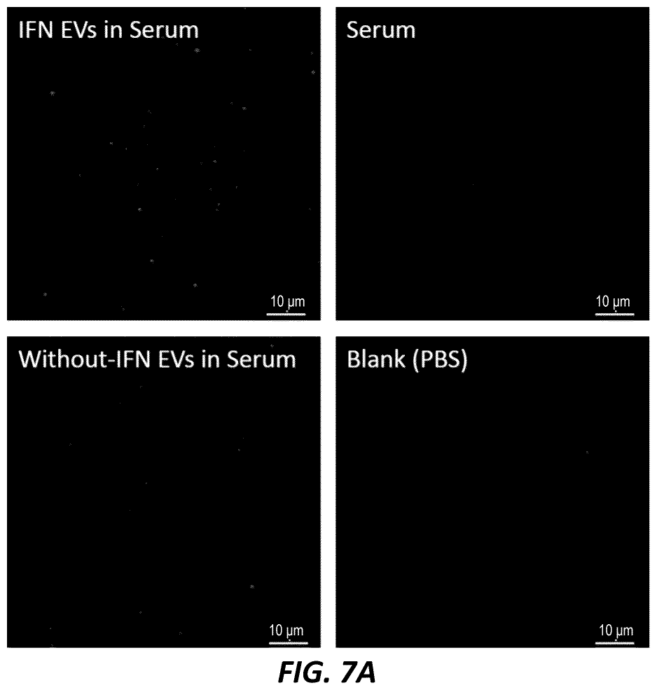

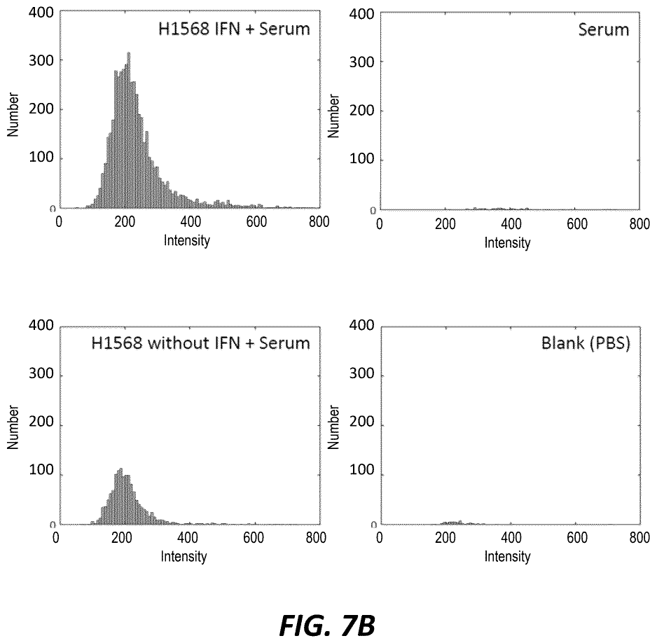

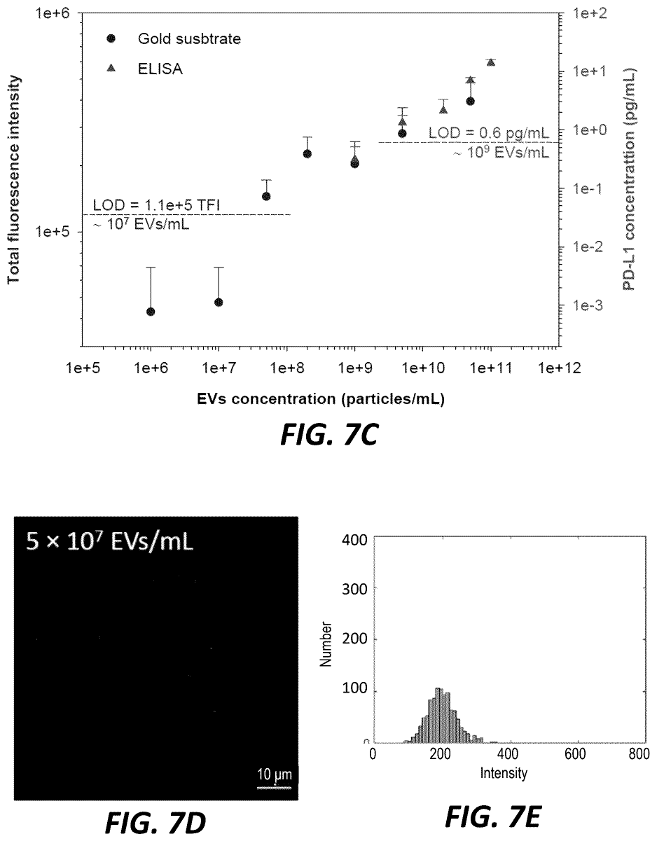

Interferon-gamma (IFN-γ), a cytokine secreted by activated effector T cells, is critical for innate and adaptive immunity, and known to upregulate PD-L1 expression on tumor cells. In the present study, IFN-γ also significantly increased the expression of H1568, an NSCLC cell line, as shown by immunofluorescence staining images (FIG. 5A) and ELISA test (p<0.001, FIG. 5B). Size distribution of EVs produced from H1568 cells with/without IFN-γ stimulation is shown in FIGS. 6A and 6B. In both cases, the majority of EVs are exosomes, which are smaller than 200 nm. These EVs were spiked in healthy donor serum at 1:1 ratio (tumor EVs: normal EVs) with 5×1010 particles/mL each to characterize their PD-L1 expression using our platform. Serum EVs at 5×1010 particles/mL and sample blank were also examined as negative controls. In order to quantify PD-L1 density on the EV membrane surface, there was no permeabilization buffer used in our EV staining procedures. TIRF images and their histograms showed that PD-L1 expressions on H1568 EVs with/without IFN-γ stimulation were successfully detected by our devices (FIGS. 7A and 7B). There were some signals in the blank control-in which PBS was used instead of EVs during the EV capture step, but all other staining steps were included. These signals might be derived from the device background and non-specific bindings of detection antibodies, which were optimized to be minimum. PD-L1 signals from tumor EV samples were remarkably higher than healthy donor serum. Additionally, PD-L1 expression on EVs of IFN-γ stimulated H1568 cells was considerably higher than EVs without stimulation (FIG. 7B). Previous studies revealed that, similar to cells, the levels of PD-L1 on tumor EVs were also upregulated by IFN-γ. As such, our platform was sensitive enough to differentiate PD-L1 levels on EVs of different samples.

We then compared the LOD of our platform with a commercialized ELISA kit. The kit that we chose was the most sensitive kit available on the market, which has a LOD of 0.6 pg/mL. EVs produced from IFN-γ stimulated H1568 cells were spiked into healthy donor serum at different concentrations from 0 to 1011 particles/mL and quantified for PD-L1 expression using our platform and ELISA. Normal EV concentration in serum was kept constant at 1011 EVs/mL for all samples. Our results showed that while ELISA could not detect PD-L1 signal in samples with tumor EV concentrations lower than 5×109 particles/mL, our platform could detect tumor EV concentrations as low as 5×107 particles/mL (FIG. 7C). At this concentration, PD-L1 signal was greater than LOD of our platform. This value was determined using SNR method, in which a SNR of three is generally accepted for estimating LOD. The noise was defined as the standard deviation of signals obtained from blanks (by three independent experiments). As such, our platform outperformed ELISA in analytical sensitivity by 102 times. Representative TIRF images of PD-L1+ EVs at capturing concentrations of 5×107 and 2×108 EVs/mL are shown in FIGS. 7D and 7E. Western blot and ELISA are the two predominant bulk immunoassays for protein analysis of EVs. In general, LOD of western blot and ELISA are 1012 and 1010 EVs/mL, respectively. There have been efforts to develop highly sensitive techniques which could compete with ELISA for EV protein quantification for medical diagnostic applications. Shao et al. labeled EVs with target-specific magnetic NPs and detected them using a miniaturized micronuclear magnetic resonance (μNMR) system. They successfully distinguished glioblastoma multiforme (GBM) derived EVs from host cell-derived EVs based on a four-GBM marker combination (EGFR, EGFRvIII, PDPN and IDH1 R132H). Using CD63-tagged EVs, they claimed that μNMR was 103-fold more sensitive than ELISA. In another study, Im et al. reported a nano-plasmonic exosome (nPLEX) assay based on transmission surface plasmon resonance through periodic nanohole arrays for label-free detection of exosomes. Using this approach, they could identify exosomes from ovarian cancer patients by their expression of CD24 and EpCAM. By measuring CD63 signal, they stated a detection sensitivity of 102-fold higher than that of chemiluminescence ELISA. Although these techniques were demonstrated to be highly sensitive for EV protein quantification, their analytical sensitivities were only examined based on signal intensity of CD63—an abundant biomarker. A validation with PD-L1 is necessary for these techniques to be considered as a diagnostic tool for immunotherapy. For PD-L1 characterization of EVs, in addition to traditional methods—western blot and ELISA, a recent study used flow cytometry to analyze EVs captured on magnetic beads, but its sensitivity was only comparable to western blot21. Lee et al. tried to capture Gli36-WT EVs on a glass slide and perform immunofluorescence staining in order to characterize conventional EV markers (tetraspanins; CD9, CD63, CD81) and tumor markers (EGFR, EGFRvIII, IDH1, IDH1R132, PDPN, PDGFRα, PD-L1, PD-L2), but failed to detect PD-L1 signal. It might be due to the low SNR of their platform, which used glass-a high background substrate. As a result, there has been intense interest in developing a highly sensitive technique for quantification of extracellular vesicular PD-L1 protein for cancer immunotherapy.

### Quantification of PD-1/PD-L1 Proteins and mRNAs From EVs of NSCLC Cancer Patients Treated With Immunotherapy

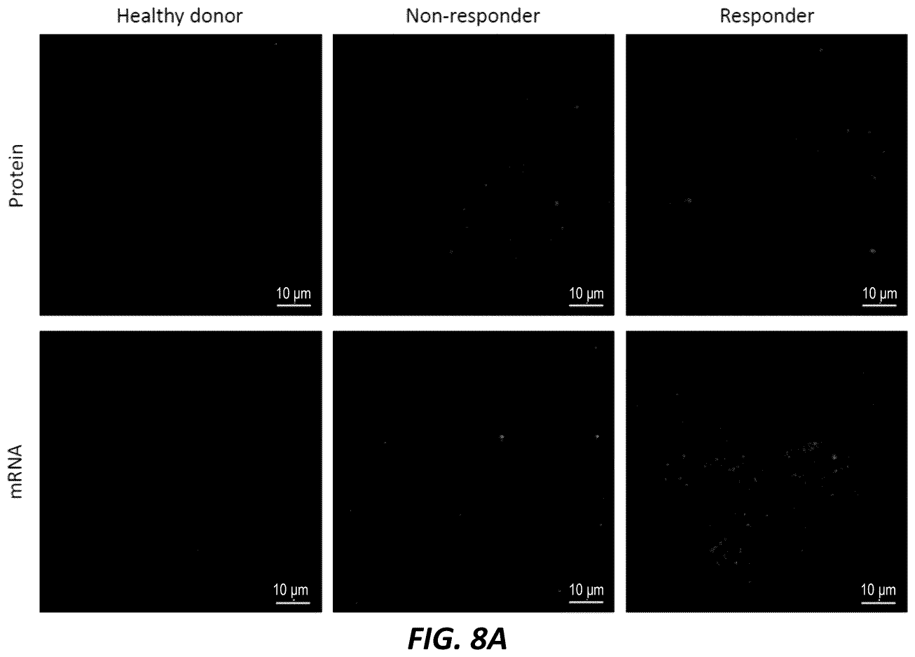

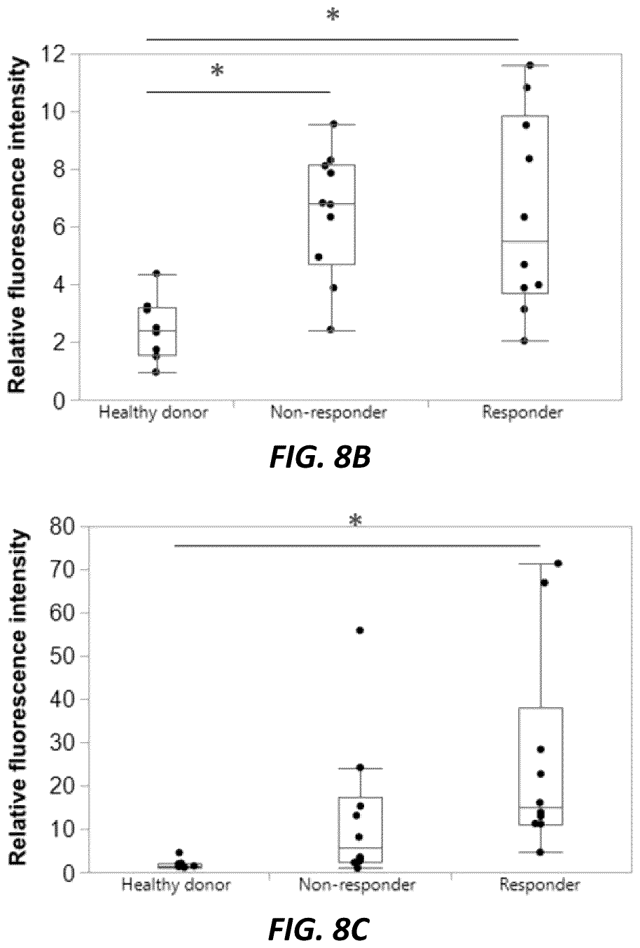

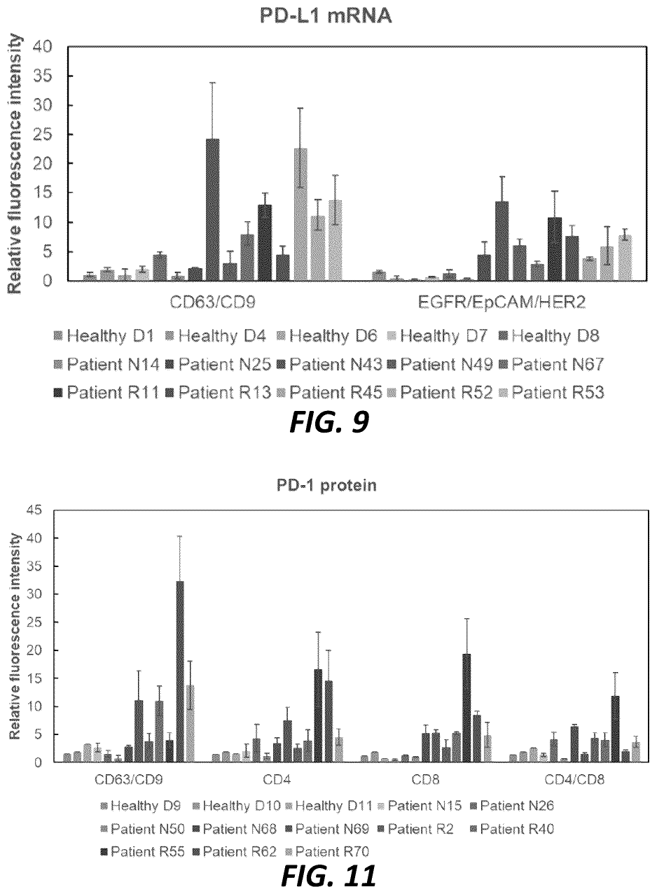

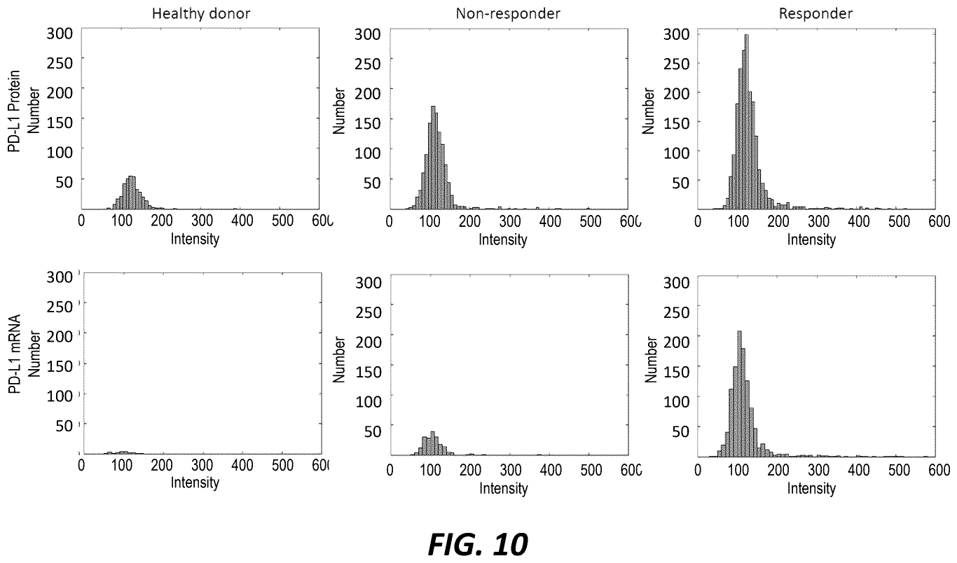

A cohort of 10 non-responders and 10 responders to immunotherapy was chosen for characterization of PD-1/PD-L1 proteins and mRNAs. Our results show that PD-L1 protein and mRNA in EVs of those patients were successfully detected using our biochip platform (FIG. 8A). The PD-L1 protein levels in patient samples (both non-responders and non-responders) were significantly higher than that of healthy donors (p<0.05, FIG. 8B). For PD-L1 mRNA levels, there was a significant difference between responders and healthy donors (p<0.05, FIG. 8C), but no significant difference between non-responders and healthy donors. In both cases, responders were well-differentiated from healthy donors, but not from non-responders. For PD-L1 protein characterization, a cocktail of EGFR, EpCAM and HER2 was selected as capture antibodies to precisely capture tumor-related EVs for further analysis because PD-L1 is known to be highly expressed in tumor cells. For characterization of PD-L1 mRNA, two different cocktails including CD63/CD9 and EGFR/EpCAM/HER2 were firstly evaluated using a cohort of 5 non-responders and 5 responders (FIG. 9). The bars in the graph of FIG. 9 represent the following patients ordered left to right and then repeated for both CD63/CD9 and EGFR/EpCAM/HER2: “Healthy D1,” “Healthy D4,” “Healthy D6,” “Healthy D7,” “Healthy D8,” “Patient N14,” “Patient N25,” “Patient N43,” “Patient N49,” “Patient N67,” “Patient R11,” “Patient R13,” “Patient R45,” “Patient R52,” and “Patient R53.” CD63 and CD9 are abundant tetraspanin surface antigens of exosomes. CD63/CD9 capture was shown to have higher PD-L1 mRNA signals than EGFR/EpCAM/HER2 capture, therefore the CD63/CD9 cocktail was used for the whole cohort of 10 non-responders and 10 responders. Representative histograms of PD-L1 protein/mRNA signals from healthy donors, non-responders and responders are presented in FIG. 10.

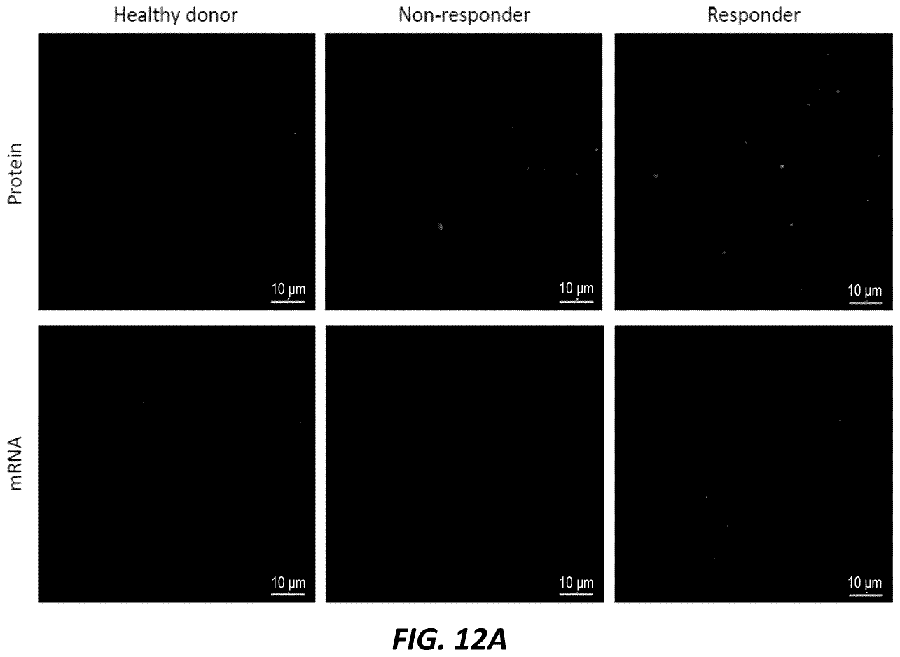

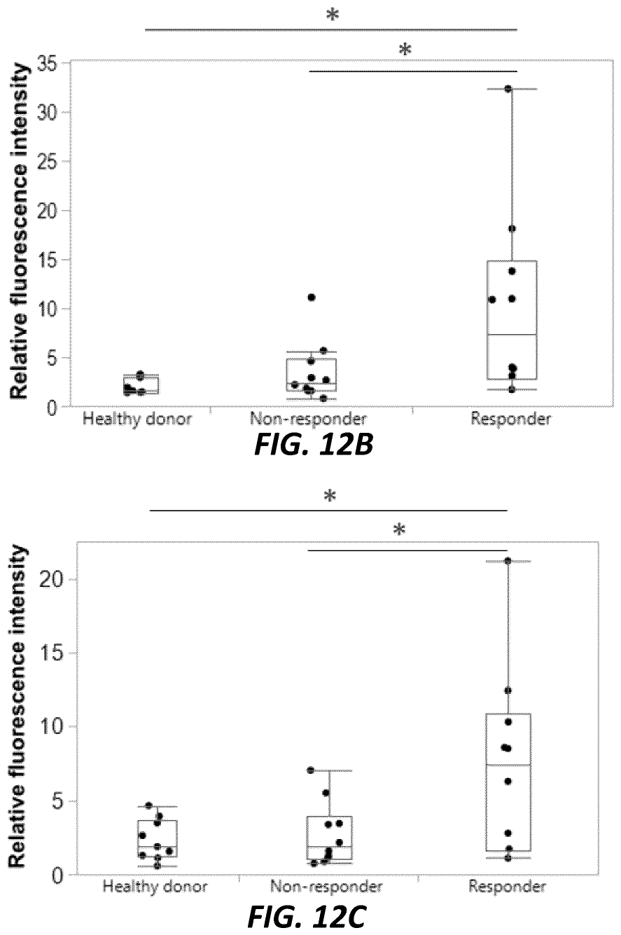

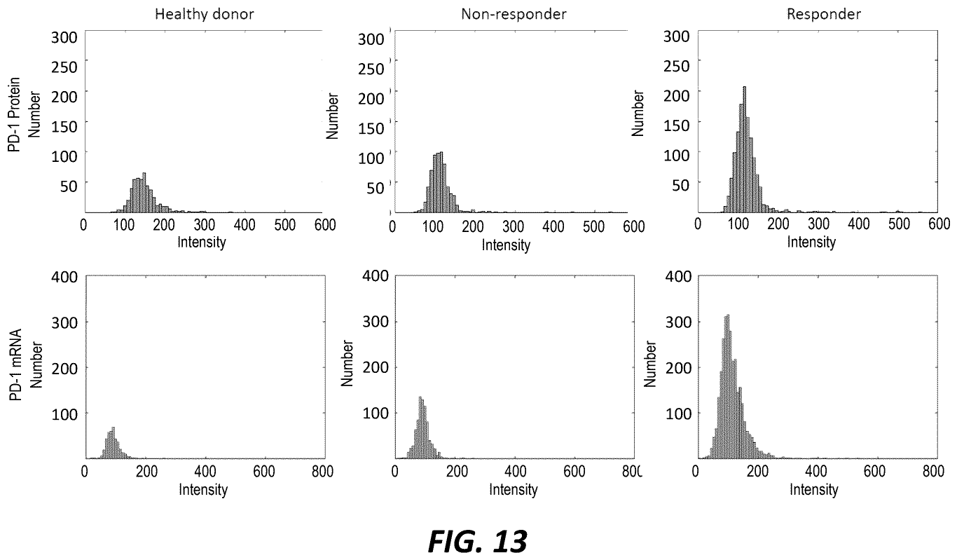

To characterize PD-1 protein in EVs, four different capture antibody cocktails, including CD63/CD9, CD4, CD8 and CD4/CD8 were firstly examined using a cohort of 5 non-responders and 5 responders (FIG. 11). Note that the bars in the graph of FIG. 11 represent the following patients ordered left to right and then repeated for CD63/CD9, CD4, CD8, and CD4/CD8: “Healthy D9,” “Healthy D10,” “Healthy D11,” “Patient N15,” “Patient N26,” “Patient N50,” “Patient N68,” “Patient N69,” “Patient R2,” “Patient R40,” “Patient R55,” “Patient R62,” and “Patient R70.” With the highest PD-1 protein signals detected on our platform, CD63/CD9 was selected as the capture cocktail for the whole cohort. In contrast to PD-L1 protein/mRNA signals, PD-1 protein levels in responders were significantly higher than both non-responders and healthy donors (p<0.05, FIG. 12A). Similar findings were observed for PD-1 mRNA with CD63/CD9 as capture antibodies (p<0.05, FIGS. 12B and 12C). Representative histograms of PD-1 protein/mRNA signals from different samples are shown in FIG. 13.

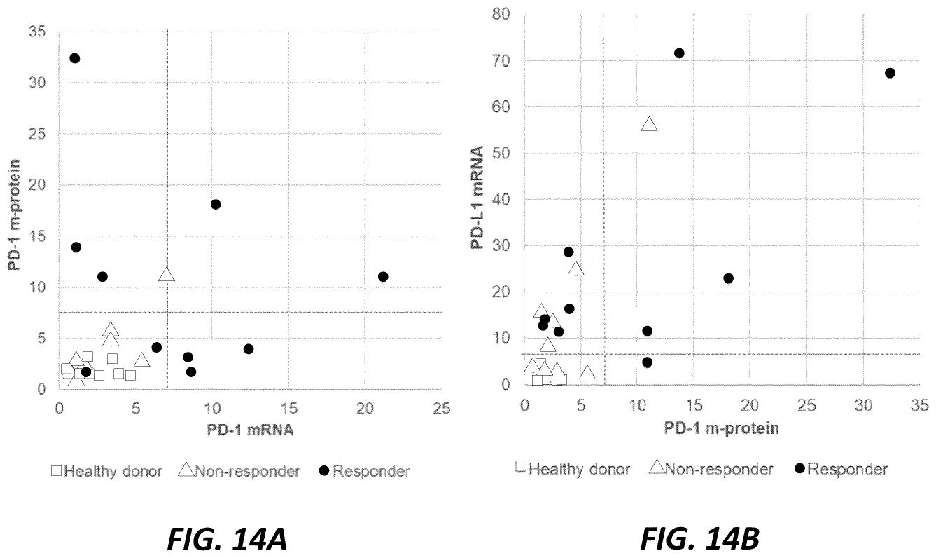

Taken together, we successfully detected and quantified PD-1/PD-L1 proteins and mRNAs in EVs purified from NSCLC serum samples. With our biochip platform, each biomarker can be accurately measured with ~3 μL serum starting volume. Scatter plots reveal that with a threshold fold change of ‘7’ for both EV mRNA and EV protein expression levels, a dual PD-1 protein/PD-1 mRNA and PD-1 protein/PD-L1 mRNA biomarkers may be helpful for identifying responders from non-responders of NSCLC patients for anti-PD-1/PD-L1 immunotherapy (FIGS. 14A and 14B). Different EV subpopulations (based on different capture antibodies) and/or more biomarkers can be characterized to well-differentiate between responders and non-responders.

Lung cancer is the second most common cancer and the most leading cause of cancer death in both men and women in the U.S., and about 80-85% of lung cancers are NSCLC (Cancer Statistics Center, American Cancer Society, 2019). Till now, FDA has approved three ICIs targeting PD-L1 (atezolizumab, durvalumab and avelumab) and two ICIs targeting PD-1 (nivolumab, pembrolizumab) for NSCLC patients with positive PD-L1 expression; however their objective response rates (ORR) were only less than 20%. This may be because PD-L1 IHC staining of tissue biopsies are not representative enough of the entire tumor burden. EVs, which are shed from primary and metastatic tumors and circulated in the bloodstream, can represent cancer heterogeneity and therefore become an ideal source for the quantification of immunotherapy biomarkers. Previous studies have shown that PD-L1 proteins are present on the surface of EVs isolated from plasma/serum of patients with metastatic melanomas, head and neck squamous cell carcinomas, gliomas, and NSCLC. In addition to PD-L1 proteins, PD-L1 mRNAs have been demonstrated to exist in EVs derived from saliva and plasma of patients with periodontitis and melanoma/NSCLC, respectively. Compared to PD-L1 proteins/mRNAs, studies on PD-1 biomarkers in EVs are limited, with only one showing the presence of PD-1 proteins on the surface of EVs. The majority of these studies used western blot, magnetic bead-based flow cytometry, RT-PCR and ddPCR to measure EV protein and mRNA levels in bulk with limited sensitivity. In this study, we therefore aimed to develop a highly sensitive technology to quantify molecular contents in EVs at the single level and simultaneously measure protein and mRNA contents on the same device.

Our biochip platform was successfully made with significantly high SNR of 482.02±21.81, which enabled us to accurately quantify low expression biomarkers of PD-1/PD-L1 proteins/mRNAs. Our platform revealed ~100 times more sensitive than ELISA in quantification of PD-L1 protein. With such high sensitivity, our technology only required ~3 μL serum starting volume for each biomarker measurement. Recently, Pang et al. claimed that with Fe3O4@TiO2 isolation and Surface-Enhanced Raman Scattering (SERS) immunoassay, they could quantify EV PD-L1 protein from 4 μL clinical serum sample and NSCLC patients could be distinguished from the healthy controls based on their PD-L1 protein expression levels. SERS has been known as an ultrasensitive analytical technique with multiplexing capability and single-molecule measurement; however, it suffers from significantly longer acquisition time than fluorescence imaging.

We successfully demonstrated a comprehensive profile of four immunotherapy biomarkers, PD-1/PD-L1 proteins on the surface and PD-1/PD-L1 mRNAs in the serum-derived EVs, of NSCLC patients. A cohort of 10 non-responders and 10 responders was examined in our study. While PD-L1 protein/mRNA levels of responders were only significantly higher than healthy donors, PD-1 protein/mRNA levels responders were shown to be significant different from both non-responders and healthy donors. The combination of dual PD-1 protein and mRNA biomarkers could therefore well-differentiate between responders and non-responders. Further investigations using larger cohorts will be performed to validate findings in the current studies. We believe that our platform has a strong potential to be developed as minimally invasive diagnostics and monitoring tool for cancer. The success of this work is a breakthrough in cancer therapy in which personalized cancer immunotherapy can be achieved by feasibly identifying patients most likely to benefit from immunotherapy and monitoring the response throughout the course of treatment.

Thus, the invention provides, among other things, a biochip for characterization of extracellular vesicular biomarkers including gold nanoparticles, methods of manufacturing the gold nanoparticle biochip, and methods for analyzing EV biomarkers using the gold nanoparticle biochip. Other features and advantages of the invention are set forth in the accompanying claims and the attached appendix.

What is claimed is:

1. A biochip for characterization of extracellular vesicular biomarkers for cancer immunotherapy, the biochip comprising:
a glass surface;
a gold film layer on the glass surface;
a plurality of gold nanoparticles coupled to the gold film layer; and
a plurality of biotinylated antibodies coupled to the gold nanoparticles, wherein the biotinylated antibodies are selected to capture extracellular vesicles.

2. The biochip of claim 1, wherein the gold film layer is coated with polyethylene glycol.

3. The biochip of claim 1, wherein the gold film layer is coated with a linker solution including at least one selected from a group consisting of a 1-thiahexa(ethyleneoxide) lipidic anchor molecule WC14, a lateral spacer β-mercaptoethanol, and biotin-polyethylene glycol (PEG)-thiol (SH).

4. The biochip of claim 1, wherein the gold film layer is coupled to the glass surface by a layer of (3-mercaptopropyl)trimethoxysilane (MPTMS).

5. The biochip of claim 1, wherein the plurality of gold nanoparticles includes a plurality of streptavidin-conjugated gold nanoparticles.

6. The biochip of claim 1, wherein each gold nanoparticle of the plurality of gold nanoparticles has a diameter of 30 nanometers.

7. An assay platform system for identifying candidates for cancer immunotherapy, the system comprising:
the biochip of claim 1; and
a total internal reflection fluorescence (TIRF) microscope imaging system configured to capture image data of extracellular vesicular biomarker captured by the biochip.

8. The system of claim 7, further comprising a controller configured to:
detect and quantify the extracellular vesicular biomarker for a first patient captured by the biochip based at least in part on image data captured by the TIRF microscope imaging system;
compare a determined quantity of the extracellular vesicular biomarker to a threshold; and
identify the first patient as a candidate for cancer immunotherapy in response to determining that the determined quantity of the extracellular vesicular biomarker exceeds the threshold.

9. A method of characterizing extracellular vesicular biomarkers for cancer immunotherapy, the method comprising:
applying purified extracellular vesicles from a biofluid of a first patient to the biochip of claim 1;
washing the biochip with a phosphate-buffered saline (PBS) solution;
applying a plurality of molecular beacons to the washed biochip;
rinsing the biochip with the PBS solution;
capturing image data of the biochip with a total internal reflection fluorescence (TIRF) microscope; and
detecting and quantifying occurrences of a first extracellular vesicular biomarker based on the captured image data.

10. The method of claim 9, wherein applying the plurality of molecular beacons to the washed biochip includes applying a plurality of cationic lipoplex nanoparticles (CLN) containing PD-1/PD-L1 target-specific molecular beacons.

11. The method of claim 9, further comprising:
comparing a determined quantity of occurrences of the first extracellular vesicular biomarker to a threshold; and
determining whether the first patient is a candidate for cancer immunotherapy based at least in part on the comparison of the determined quantity of occurrences of the first extracellular vesicular biomarker and the threshold.

12. A method for evaluating anti-PD-L1/PD-1 immunotherapy comprising coupling CD63/CD9 to a biochip of claim 1 as the capture antibodies to capture EV-based PD-L1 and PD-1 mRNA and membrane proteins from a liquid biopsy sample.

13. A method for evaluating an immunotherapy, the method comprising coupling at least one capture antibody to the biochip of claim 1, wherein the at least one capture antibody is selected to capture from a liquid biopsy sample at least one selected from a group consisting of a mRNA and a protein indicative of a response to the immunotherapy.

14. A method for evaluating anti-PD-L1/PD-1 immunotherapy comprising using CD63/CD9 rich EV-based PD-L1 and PD-1 mRNA/membrane protein as a liquid biopsy biomarker for anti-PD-L1/PD-1 immunotherapy.
